(19) United States
(12) Patent Application Publication
(10) Pub. No.: US 2022/0008254 A1
(43) Pub. Date: Jan. 13, 2022

(54) POST-OPERATIVE FOAM AND SUPER-ABSORBENT DRESSING

(71) Applicant: KCI LICENSING, INC., San Antonio, TX (US)

(72) Inventor: Christopher B. LOCKE, Bournemouth (GB)

(21) Appl. No.: 17/294,185

(22) PCT Filed: Nov. 12, 2019
(86) PCT No.: PCT/US2019/060944

§ 371 Date: May 14, 2021

Related U.S. Application Data
(60) Provisional application No. 62/769,234, filed on Nov. 19, 2018.

(51) Int. Cl. A61F 13/02

(52) U.S. Cl. CPC A61F 13/0209; A61F 2013/0074; A61F 13/0289; A61F 13/022

(57) ABSTRACT

A dressing includes a hydrophilic foam layer having a longitudinal central axis configured to overlay the incision, a drape coupled to an outwardly-facing side of the hydrophilic foam layer, a first row of superabsorbent material positioned on the outwardly-facing side of the hydrophilic foam layer, and a second row of superabsorbent material positioned on the outwardly-facing side of the hydrophilic foam layer. The first row and the second row are spaced laterally apart from, and on opposite sides of, the longitudinal central axis. The drape includes a first window aligned with the first row and a second window aligned with the second row. The first window and the second window include a film, different from the drape, configured to facilitate evaporation therethrough.

### CROSS-REFERENCE TO RELATED APPLICATIONS

This application claims the benefit of priority to U.S. Provisional Application No. 62/769,234, filed on Nov. 19, 2018, which is incorporated herein by reference in its entirety.

### BACKGROUND

The present disclosure relates generally to the field of dressings for treating wounds, and more particularly to dressings for treating incisions following surgical operations. Surgical operations often require incisions through a patient's skin and other tissue. At the conclusion of such an operation, the incision is typically sutured closed, leaving a suture line along which the incision may heal. While the incision heals, the incision may exude fluid. Healing of the wound may be improved if at least some of this fluid is removed from the incision and the surrounding periwound area. Accordingly, post-operative treatment of an incision may benefit from a dressing suitable to treating elongated wounds while wicking fluid away from the incision.

### SUMMARY

One implementation of the present disclosure is a dressing for use with an incision. The dressing includes a hydrophilic foam layer having a longitudinal central axis configured to overlay the incision, a drape coupled to an outwardly-facing side of the hydrophilic foam layer, a first row of superabsorbent material positioned on the outwardly-facing side of the hydrophilic foam layer, and a second row of superabsorbent material positioned on the outwardly-facing side of the hydrophilic foam layer. The first row and the second row are spaced laterally apart from, and on opposite sides of, the longitudinal central axis. The drape includes a first window aligned with the first row and a second window aligned with the second row. The first window and the second window include a film, different from the drape, configured to facilitate evaporation therethrough.

In some embodiments, the first row and the second row provide lateral wicking of a fluid from the longitudinal central axis to the first row and the second row.

In some embodiments, the dressing includes a hydrophobic film layer positioned along a wound-facing side of the hydrophilic foam layer. In some embodiments, the hydrophobic film layer includes an opening extending along the longitudinal central axis, the opening configured to allow fluid to flow therethrough from the wound to the hydrophilic foam layer. In some embodiments, the hydrophobic film layer includes a pattern-printed film at least partially aligned with the first row and the second row and configured to at least partially prevent the flow of fluid from the hydrophilic foam to a periwound area around the incision.

In some embodiments, the hydrophilic foam layer provides a storage reservoir for the fluid. The first row and the second row establish an evaporative gradient flow path for the fluid from the longitudinal central axis through the first window and the second window.

In some embodiments, the dressing includes a non-woven fibrous layer positioned between the hydrophilic foam layer and the drape and configured to provide enhanced lateral wicking of fluid away from the longitudinal central axis and toward the first and second rows.

In some embodiments, the dressing includes a first section of fuseable fiber configured to couple the first row to the drape and a second section of fuseable fiber configured to couple the second row to the drape.

In some embodiments, the first row includes a plurality of non-contiguous printed deposits of the superabsorbent material. In some embodiments, the deposits include dots that are at least one of circular or elliptical. In some embodiments, the first row and the second row are substantially parallel to the longitudinal central axis. In some embodiments, the dressing is substantially symmetrical across the longitudinal central axis.

In some embodiments, the film has a higher moisture vapor transmission rate than the drape.

Another implementation of the present disclosure is a method manufacturing a dressing. The method includes providing a hydrophilic foam layer having a longitudinal central axis and printing a first row of superabsorbent material and a second row of superabsorbent material on the hydrophilic foam layer. The first row and the second row are spaced apart from the longitudinal central axis. The method also includes forming a drape having a first film window and a second film window configured to allow evaporation therethrough and coupling the drape to the hydrophilic foam layer with the first film window aligned with the first row and the second film window aligned with the second row.

In some embodiments, the method includes positioning a hydrophobic film layer along the hydrophilic foam layer such that the hydrophilic foam layer is between the hydrophobic film layer and the drape.

In some embodiments, printing the first row of superabsorbent material comprises depositing a superabsorbent slurry in a pattern to form the first row. In some embodiments, the method includes coupling the first row and the second row to the drape with a fusible fiber.

Another implementation of the present disclosure is a method of treating an incision. The method includes aligning a longitudinal central axis of a hydrophilic foam layer with the incision, absorbing, by the hydrophilic foam layer, fluid from the incision, and laterally wicking the fluid through the hydrophilic foam layer to a first row of superabsorbent material and a second row of superabsorbent material. The first row and the second row spaced apart from and on opposite sides of the longitudinal central axis. The method also includes allowing evaporation of the fluid from the first row and the second row through windows in a drape coupled to the hydrophilic foam layer, the windows overlying the rows.

In some embodiments, the method includes allowing the fluid to flow from the incision to the hydrophilic foam layer via an opening in a hydrophobic film layer coupled to a wound-facing side of the hydrophilic foam layer, and at least partially preventing, by the hydrophobic film layer, exposure of a periwound area to the fluid.

### BRIEF DESCRIPTION OF THE DRAWINGS

FIG. 1 is a perspective view of a dressing for treating an incision, according to an exemplary embodiment.

FIG. 2 is exploded, cross-sectional view of the dressing of FIG. 1, according to an exemplary embodiment.

FIG. 3 is a top view of the dressing of FIG. 1 with a first pattern of superabsorbent rows, according to an exemplary embodiment.

FIG. 4 is a top view of the dressing of FIG. 1 with a second pattern of superabsorbent rows, according to an exemplary embodiment.

FIG. 5 is a top view of the dressing of FIG. 1 with a third pattern of superabsorbent rows, according to an exemplary embodiment.

### DETAILED DESCRIPTION

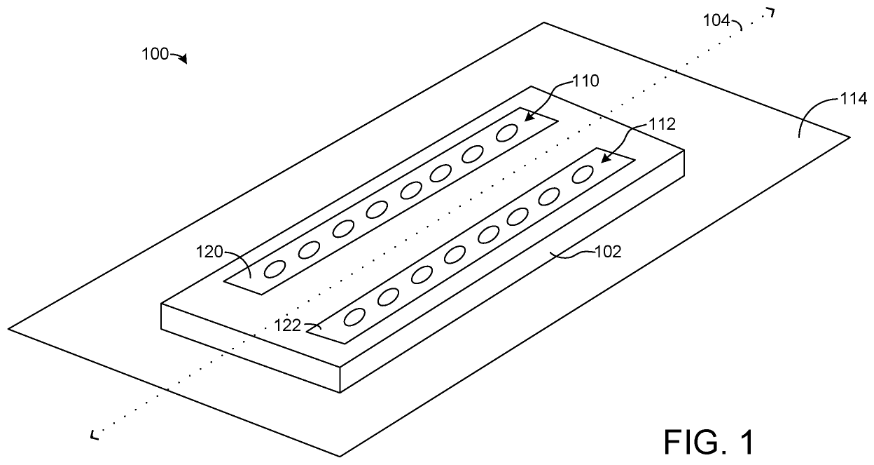

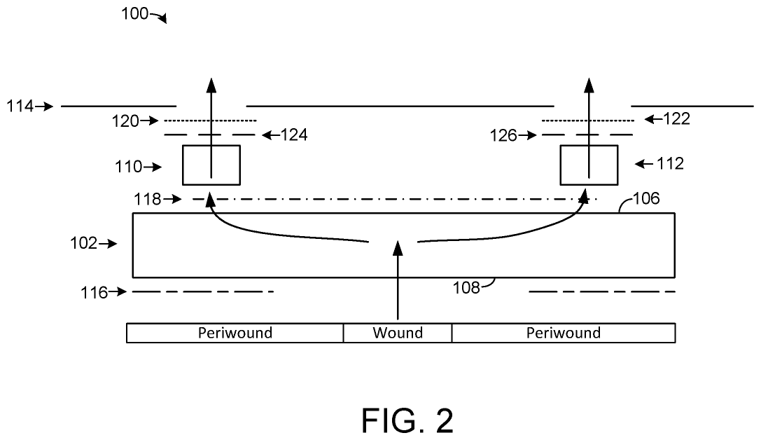

Referring to FIGS. 1-2, a dressing 100 is shown, according to an exemplary embodiment. FIG. 1 shows a perspective view of the dressing 100 while FIG. 2 shows an exploded, cross-sectional view of the dressing 100. The dressing 100 is configured for treating an elongated wound. The elongated wound may be a surgical incision, or cut, sore, burn, wound, etc. The wound may be sutured, stitched, stapled, glued, etc. along the incision before application of the dressing 100. A periwound area surrounds the wound. The dressing 100 is configured to provide lateral wicking of fluid away from the wound, evaporation of the fluid, and at least partial protection of the periwound area from exposure to the fluid.

The dressing 100 includes a hydrophilic foam layer 102 having a longitudinal central axis 104, an outwardly-facing side 106, and an inwardly-facing side 108. The dressing 100 also includes a first row 110 of superabsorbent material positioned on the outwardly-facing side 106, a second row 112 of superabsorbent material positioned on the outwardly-facing side 106, and a drape 114 coupled to the outwardly-facing side 106 of the hydrophilic foam layer 102. In some embodiments, the dressing 100 also includes a hydrophobic film layer 116 coupled to the inwardly-facing side 108 of the hydrophilic foam layer 102. In some embodiments, the dressing 100 also includes a non-woven fibrous layer 118 positioned between the hydrophilic foam layer 102 and the drape 114.

The hydrophilic foam layer 102 is configured to absorb fluid from the wound. The hydrophilic foam layer 102 may be made of a highly hydrophilic foam, for example as commercially available from AMS or Freudenburg and used in the TIELLE ESSENTIAL™ Foam Dressings by ACELITY™. The inwardly-facing side 108 of the hydrophilic foam layer 102 is configured to be placed along a wound, while the outwardly-facing side 106 is opposite the inwardly-facing side 108 and directed away from the wound.

The hydrophilic foam layer 102 may be substantially rectangular and may be bisected by the longitudinal central axis 104. The hydrophilic foam layer 102 may have a length in the direction of the longitudinal central axis 104 substantially longer than a width of the hydrophilic foam layer 102 (i.e., orthogonal to the longitudinal central axis 104), such that the dressing 100 is shaped to fit over an elongated wound (e.g., incision).

In some embodiments, a visual indicator is included on the dressing 100 (e.g., on the inwardly-facing side 108 of the hydrophilic foam layer 102, on the drape 114) indicating the location of the longitudinal central axis 104, for example to indicate that the longitudinal central axis 104 should be aligned with the wound when applying the dressing 100. In some embodiments, a pair of printed lines, parallel to and positioned on opposite sides of the longitudinal central axis 104, are included on the inwardly-facing side 108 to indicate a region where the wound preferably contacts the dressing 100.

The first row 110 of superabsorbent material and the second row 112 of superabsorbent material are positioned on the outwardly-facing side 106 of the hydrophilic foam layer 102. The first row 110 and the second row 112 are laterally spaced apart from the longitudinal central axis 104 and are positioned on opposite sides of the longitudinal central axis 104. The first row 110 and the second row 112 may be substantially parallel to the longitudinal central axis 104. Accordingly, the first row 110 and the second row 112 are positioned away from the area of the hydrophilic foam layer 102 intended to be positioned along an incision. In some embodiments, one or more additional rows or other collections of superabsorbent material are positioned on the hydrophilic foam layer 102, preferably spaced away from the longitudinal central axis 104.

The first row 110 and the second row 112 are configured to absorb fluid from the hydrophilic foam layer 102. The superabsorbent material of the first row 110 and the second row 112 may be more hydrophilic than the hydrophilic foam layer 102. The first row 110 and the second row 112 may thereby provide lateral wicking of fluid from the longitudinal central axis 104 towards the first row 110 and the second row 112 (i.e., away from an incision/wound substantially aligned with the longitudinal central axis 104, out of the hydrophilic foam layer 102). The first row 110 and the second row 112 also provide storage of fluid away from the wound and the periwound, for example to protect tissue from maceration.

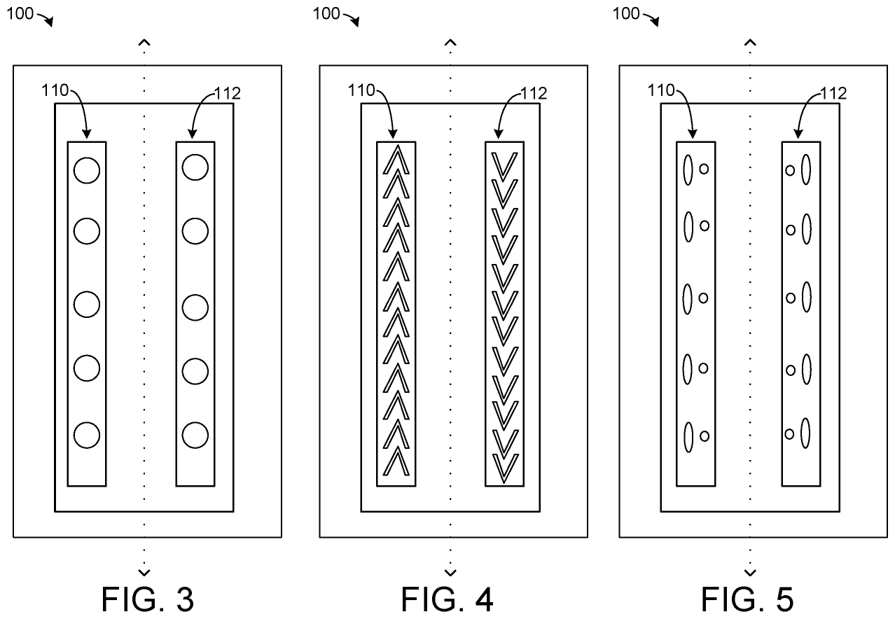

To create the first row 110 and the second row 112 on the hydrophilic foam layer 102, a slurry of superabsorbent material is deposited (e.g., printed) onto the hydrophilic foam layer 102. For example, the first row 110 and the second row 112 are made up of a dried slurry of BASF Luquasorb or other superabsorbent granules (e.g., sodium polyacrylate, polyacrylamide copolymer, ethylene maleic anhydride copolymer, cross-linked carboxymethylcellulose, polyvinyl alcohol copolymers, cross-linked polyethylene oxide). In some embodiments, the superabsorbent material includes a dye released when fluid is absorbed, for example to provide a visual indication of fluid in the dressing 100. The superabsorbent material may be printed onto the hydrophilic foam layer 102 in a variety of patterns in various embodiments, for example as illustrated in FIGS. 3-5 and described in detail with reference thereto. The first row 110 and the second row 112 may each include a series of deposits (dots, lines, shapes, etc.) that form a pattern of superabsorbent material. In preferred embodiments, the deposits are spaced-apart and shaped such that the deposits are not joined together, even when the superabsorbent material absorbs fluid and swells to a larger size. Such a pattern of superabsorbent in the first row 110 and the second row 112 may improve flexibility and conformability of the dressing 100, even after the superabsorbent absorbs fluid from the wound and/or the fluid evaporates from the superabsorbent.

The drape 114 is coupled to the hydrophilic foam layer 102, the first row 110, and the second row 112 along the outwardly-facing side 106 of the hydrophilic foam layer 102. In some embodiments, the drape 114 includes an adhesive on an inside surface of the drape 114 configured to couple the drape 114 to the hydrophilic foam layer 102, the first row 110, and the second row 112 and/or the patient's skin (i.e., to a periwound area around the incision). In some embodiments, a border adhesive configured to couple the dressing 100 to the periwound may include a hydrocolloid or silicone adhesive, for example as included in DERMATAC™ Film adhesive by ACELITY™. The drape 114 may comprise a material configured to substantially prevent external contaminants from entering the dressing 100.

The drape 114 includes a first window 120 aligned with the first row 110 and a second window 122 aligned with the second row 112. The first window 120 and the second window 122 may be laterally spaced apart from the longitudinal central axis 104 and positioned on opposite sides of the longitudinal central axis 104. In some embodiments, a first section 124 of fuseable fiber couples the first row 110 to the drape 114 (e.g., to the first window 120) and a second section 126 of fuseable fiber couples the second row 112 to the drape 114 (e.g., to the second window 122). In various embodiments, various numbers of windows may be included.

At the first window 120 and the second window 122, the material of the remainder of the drape 114 is replaced by a film configured to facilitate evaporation therethrough. The film of the windows 120, 122 may have a higher moisture vapor transfer rate (MVTR) than the drape 114. In some embodiments, the film is flexible to allow expansion of the superabsorbent material when the superabsorbent material absorbs fluid and swells, while also substantially ensuring proximity of the film to the superabsorbent material when the superabsorbent material is not swollen. The first window 120 and the second window 122 thereby provide for evaporation of fluid from the first row 110 and second row 112 of superabsorbent material.

The first window 120 and the second window 122 thereby contribute to an evaporative gradient flow path through the dressing 100. That is, as illustrated by arrows in FIG. 2, the hydrophilic foam layer 102 provides a storage reservoir for fluid absorbed from the wound. The first row 110 and the second row 112 of superabsorbent material establish an evaporative gradient flow path for the fluid from the longitudinal central axis 11 and through the first window 120 and the second window 122. The relatively low MVTR of the drape 114 compared to the MVTR of the first window 120 and the second window 122 may facilitate the fluid in flowing laterally to the first row 110 and the second 112 before evaporation. The gradient flow path and lateral wicking of the dressing 100 may facilitate distribution of fluid through the dressing 100 to maximize the percentage of the volume of dressing 100 used for fluid handling. Evaporation and any agitation caused by the evaporation is thereby laterally separated from the wound, protecting the vulnerable tissue while also first removing fluid from the periwound area to protect the periwound from maceration. Furthermore, by establishing two linear areas of the dressing 100 that provide for evaporation, the rate of evaporation from the dressing 100 may be substantially higher than for a traditional dressing where evaporation is limited to a single area above the wound.

In alternative embodiments, the drape 114 includes a single window of high-MVTR film positioned over the hydrophilic foam layer 102 and a border positioned around the single window and configured to bind the dressing 100 to the periwound. In such a case, a fuseable fiber layer may be used to bind the hydrophilic foam layer 102 to the drape 114.

In some embodiments, for example as illustrated in FIG. 2, a fibrous layer 118 is included between the hydrophilic foam layer 102 and the drape 114. The fibrous layer 118 is configured to provide enhanced lateral wicking of fluid away from the longitudinal central axis 104 laterally to the first row 110 and the second row 112. The fibrous layer 118 may be made of a non-woven fibrous material configured for a high rate of fluid wicking and/or to provide fluid wicking in a particular direction.

In some embodiments, a hydrophobic film layer 116 is positioned at the inwardly-facing side 108 of the hydrophilic foam layer 102 and is configured to manage the flow of fluid between the hydrophilic foam layer 102 and the wound and/or periwound.

The hydrophobic film layer 116 includes a pattern-printed film having a first panel 128 at least partially aligned with the first row 110 and a second panel 130 at least partially aligned with the second row 112, i.e., such that the first panel 128 and the second panel 130 are spaced laterally apart from the longitudinal central axis 104 and positioned on opposite sides of the longitudinal central axis 104. The pattern-printed film (i.e., the first panel 128 and the second panel 130) is configured to at least partially prevent the flow of fluid from the hydrophilic foam layer 102 to a periwound area around the wound/incision. For example, the pattern-printed film may be approximately 50% blocking to fluids. The pattern-printed film may thereby discourage exposure of the periwound to fluid while still allowing fluid to be absorbed into the hydrophilic foam layer 102 through the first panel 128 and the second panel 130.

The hydrophobic film layer 116 includes an opening 132 extending along the longitudinal central axis 104 and positioned between the first panel 128 and the second panel 130. The opening 132 is configured to allow fluid to flow therethrough from the wound to the hydrophilic foam layer 102. That is, in a preferred application, the opening 132 is aligned with the wound such that the wound may be in contact with the hydrophilic foam layer 102 through the opening 132. In some embodiments, the opening 132 is sized to be approximately 50% of the size of the inwardly-facing side 108 of the hydrophobic foam layer, with the first panel 128 and the second panel 130 sized to substantially cover the remaining approximately 50%. Various other relative dimensions are possible in various embodiments.

The hydrophobic film layer 116 (i.e., the first panel 128, the opening 132, and the second panel 130) is thereby configured to encourage absorption of fluid proximate the longitudinal central axis 104, while at least partially preventing the fluid from being distributed to the periwound by the lateral wicking of the dressing 100. Accordingly, the hydrophobic film layer 116 allows the dressing 100 to provide beneficial lateral wicking while minimizing a risk of maceration or other complication due to fluid exposure at the periwound.

Referring now to FIGS. 3-5, top views of various embodiments of the dressing 100 have various patterns of superabsorbent material in the first row 110 and the second row 112 are shown, according to exemplary embodiment. As illustrated by FIGS. 3-5, the superabsorbent material may be deposited (e.g., printed) onto the hydrophilic foam layer 102 in a variety of patterns or shapes. Each row 110, 112 may include multiple deposits of superabsorbent material. In preferred embodiments, the deposits are spaced apart (i.e., not formed together) to allow flexibility and conformability of the dressing 100, to allow the dressing 100 to stretch, etc. The spacing, sizing, shaping, etc. of the deposits also preferably prevents the deposits from joining to form a continuous mass when swollen with fluid. This allows the dressing 100 to return to a flexible, conformable state after fluid evaporates from the superabsorbent material.

In the example of FIG. 3, the deposits are substantially circular. In other embodiments, the deposits may be elliptical. In the example of FIG. 4, the deposits form chevron shapes. In the example of FIG. 5, the first row 110 and the second row 112 both include pairs of deposits, such that the embodiment of FIG. 5 may be characterized as including four rows of superabsorbent material. In various embodiments, various numbers of rows of superabsorbent material is included. It should be understood that the examples included herein are for example purposes and that many other patterns of superabsorbent deposits are possible.

Although the embodiments of the dressing 100 described herein focus on lateral wicking of fluid away from a substantially linear wound/incision, it should be understood that the present disclosure also contemplates similar embodiments for other geometries (e.g., round, curved, etc.) and/or other desired fluid flow paths. In other words, by selecting the relative location and shape of the superabsorbent materials, windows of high MVTR film, panels/openings in a hydrophobic film layer, etc. as described in detail above, various wicking/gradient flow paths may be established to provide fluid management as desired for various applications. In the embodiment shown, the dressing 102 is substantially symmetrical across the longitudinal central axis. In other embodiments, the dressing 102 may be asymmetrical. In some embodiments, the dressings contemplated herein may be configured for use with negative pressure wound therapy.

As utilized herein, the terms “approximately,” “about,” “substantially,” and similar terms are intended to have a broad meaning in harmony with the common and accepted usage by those of ordinary skill in the art to which the subject matter of this disclosure pertains. It should be understood by those of skill in the art who review this disclosure that these terms are intended to allow a description of certain features described and claimed without restricting the scope of these features to the precise numerical ranges provided. Accordingly, these terms should be interpreted as indicating that insubstantial or inconsequential modifications or alterations of the subject matter described and are considered to be within the scope of the disclosure.

The term “coupled” and variations thereof, as used herein, means the joining of two members directly or indirectly to one another. Such joining may be stationary (e.g., permanent or fixed) or moveable (e.g., removable or releasable). Such joining may be achieved with the two members coupled directly to each other, with the two members coupled to each other using a separate intervening member and any additional intermediate members coupled with one another, or with the two members coupled to each other using an intervening member that is integrally formed as a single unitary body with one of the two members. If “coupled” or variations thereof are modified by an additional term (e.g., directly coupled), the generic definition of “coupled” provided above is modified by the plain language meaning of the additional term (e.g., “directly coupled” means the joining of two members without any separate intervening member), resulting in a narrower definition than the generic definition of “coupled” provided above. Such coupling may be mechanical, electrical, or fluidic.

Other arrangements and combinations of the elements described herein and shown in the Figures are also contemplated by the present disclosure. The construction and arrangement of the systems and apparatuses as shown in the various exemplary embodiments are illustrative only. Although only a few embodiments have been described in detail in this disclosure, many modifications are possible (e.g., variations in sizes, dimensions, structures, shapes and proportions of the various elements, values of parameters, mounting arrangements, use of materials, colors, orientations, etc.). For example, the position of elements can be reversed or otherwise varied and the nature or number of discrete elements or positions can be altered or varied. Accordingly, all such modifications are intended to be included within the scope of the present disclosure. Other substitutions, modifications, changes, and omissions can be made in the design, operating conditions and arrangement of the exemplary embodiments without departing from the scope of the present disclosure.

What is claimed is:

1. A dressing for use with an incision, comprising:
a hydrophilic foam layer having a longitudinal central axis configured to overlay the incision;
a drape coupled to an outwardly-facing side of the hydrophilic foam layer;
a first row of superabsorbent material positioned on the outwardly-facing side of the hydrophilic foam layer; and
a second row of superabsorbent material positioned on the outwardly-facing side of the hydrophilic foam layer;
wherein the first row and the second row are spaced laterally apart from, and on opposite sides of, the longitudinal central axis; and
wherein the drape comprises a first window aligned with the first row and a second window aligned with the second row, the first window and the second window comprising a film, different from the drape, configured to facilitate evaporation therethrough.

2. The dressing of claim 1, wherein the hydrophilic foam layer is configured to absorb a fluid from an incision.

3. The dressing of claim 1, wherein the first row and the second row provide lateral wicking of a fluid from the longitudinal central axis to the first row and the second row.

4. The dressing of claim 1, further comprising a hydrophobic film layer positioned along a wound-facing side of the hydrophilic foam layer.

5. The dressing of claim 4, wherein the hydrophobic film layer comprises an opening extending along the longitudinal central axis, the opening configured to allow fluid to flow therethrough from the wound to the hydrophilic foam layer.

6. The dressing of claim 5, wherein the hydrophobic film layer comprises a pattern-printed film at least partially aligned with the first row and the second row and configured to at least partially prevent the flow of fluid from the hydrophilic foam layer to a periwound area around the incision.

7. The dressing of claim 6, wherein the hydrophilic foam layer provides a storage reservoir for the fluid; and
wherein the first row and the second row establish an evaporative gradient flow path for the fluid from the longitudinal central axis through the first window and the second window.

8. The dressing of claim 1, comprising a non-woven fibrous layer positioned between the hydrophilic foam layer and the drape and configured to provide enhanced lateral wicking of fluid away from the longitudinal central axis and toward the first and second rows.

9. The dressing of claim 1, comprising a first section of fuseable fiber configured to couple the first row to the drape and a second section of fuseable fiber configured to couple the second row to the drape.

10. The dressing of claim 1, wherein the first row comprises a plurality of non-contiguous printed deposits of the superabsorbent material.

11. The dressing of claim 10, wherein the deposits comprise dots that are at least one of circular or elliptical.

12. The dressing of claim 1, wherein the first row and the second row are substantially parallel to the longitudinal central axis.

13. The dressing of claim 1, wherein the dressing is substantially symmetrical across the longitudinal central axis.

14. The dressing of claim 1, wherein the film has a higher moisture vapor transmission rate than the drape.

15. A method of manufacturing a dressing, comprising:
providing a hydrophilic foam layer having a longitudinal central axis;
printing a first row of superabsorbent material and a second row of superabsorbent material on the hydrophilic foam layer, the first row and the second row spaced apart from the longitudinal central axis;
forming a drape having a first film window and a second film window configured to allow evaporation therethrough; and
coupling the drape to the hydrophilic foam layer with the first film window aligned with the first row and the second film window aligned with the second row.

16. The method of claim 15, comprising positioning a hydrophobic film layer along the hydrophilic foam layer such that the hydrophilic foam layer is between the hydrophobic film layer and the drape.

17. The method of claim 16, comprising providing the hydrophobic film layer with an opening and aligning the opening with the longitudinal central axis of the hydrophobic film layer.

18. The method of claim 15, wherein printing the first row of superabsorbent material comprises depositing a superabsorbent slurry in a pattern to form the first row.

19. The method of claim 15, comprising coupling the first row and the second row to the drape with a fuseable fiber.

20. A method of treating an incision, comprising:
aligning a longitudinal central axis of a hydrophilic foam layer with the incision;
absorbing, by the hydrophilic foam layer, fluid from the incision;
laterally wicking the fluid through the hydrophilic foam layer to a first row of superabsorbent material and a second row of superabsorbent material, the first row and the second row spaced apart from and on opposite sides of the longitudinal central axis;
allowing evaporation of the fluid from the first row and the second row through windows in a drape coupled to the hydrophilic foam layer, the windows overlying the rows.

21. The method of claim 20, comprising:
allowing the fluid to flow from the incision to the hydrophilic foam layer via an opening in a hydrophobic film layer coupled to a wound-facing side of the hydrophilic foam layer;
at least partially preventing, by the hydrophobic film layer, exposure of a periwound area to the fluid.
